(19) United States
(12) Patent Application Publication
(10) Pub. No.: US 2019/0357762 A1
(43) Pub. Date: Nov. 28, 2019

(54) MODULAR WIRELESS LARGE BORE VACUUM UNIVERSAL ENDOSCOPE AND VACUUMSCOPE

(71) Applicant: The Regents of the University of California, Oakland, CA (US)

(72) Inventors: Ralph V. Clayman, Orange, CA (US); Kamaljot S. Kaler, Orange, CA (US); Andrew E. Heidari, Irvine, CA (US)

(21) Appl. No.: 16/421,061

(22) Filed: May 23, 2019

Related U.S. Application Data
(60) Provisional application No. 62/675,929, filed on May 24, 2018.

(51) Int. Cl. A61B 1/307; A61B 1/00; A61B 1/06; A61M 1/00

(52) U.S. Cl. CPC A61B 1/307; A61B 1/00094; A61B 2017/22079; A61B 1/00119; A61M 1/0023; A61B 1/0676

(57) ABSTRACT

The present technology relates to ureteroscopy, laser ablation of ureteral and renal stone, capture and removal of stone fragments. In one embodiment, the device includes an optical instrument operably connected to a large vacuum channel between 1.5 mm and 8.0 mm in width. In another embodiment, the device includes two single-time use disposable or potentially reusable units, such as a large vacuum endoscope removal tip and wireless and modular battery powered hand piece.

### CROSS-REFERENCE TO RELATED APPLICATIONS

The present application claims the benefit of priority under 35 U.S.C. § 119(e) of provisional application Ser. No. 62/675,929, filed May 24, 2018, the contents of which are hereby incorporated by reference.

### FIELD OF THE INVENTION

The present disclosure relates to the medical field, and more specifically, ureteroscopy and surgical procedures.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Nephrolithiasis, or the presence of renal calculi (kidney stones), is a common health problem across the globe with a prevalence of 10%. The prevalence is increasing given the current obesity epidemic. Nephrolithiasis may lead to persistent renal obstruction and permanent renal damage if left unresolved. On an annual basis the cost to the USA economy ($10 billion) for the care and treatment of urolithiasis is greater than for any other urological condition. The failure to completely render the stone bearing kidney free of all stone material results in a high rate of recurrent stone disease and further patient debility and expense.

The current acute treatment options for small renal/ureteral stones (i.e. </=7 mm in diameter) nephrolithiasis includes pain medication and hydration until the stone passes naturally or with pharmaceutical interventions that facilitate stone passage; approximately half of these small stones (<5 mm) will pass without the need for intervention. In instances where this approach fails or for larger stones, patients are treated with shock wave lithotripsy or ureteroscopic lithotripsy with electrohydraulic or laser probes as first-line management options, or percutaneous nephrolithotomy or laparoscopic stone removal when the stone is large (i.e. >1.5 cm). In contrast to shock wave lithotripsy, ureteroscopy is a less expensive procedure, yields higher stone-free rates, and can treat complicated, dense, or larger renal calculi up to 1.5 cm in diameter, in which shock wave lithotripsy may be unsuccessful. Recent advances in ureteroscopy have also reduced complication rates. Of note, the major drawback to both shock wave lithotripsy as well as ureteroscopy is that the stone clearance rate is no better than 50% on computed tomography (CT) scans; leaving even small stone fragments behind, puts the patient at high risk for recurrent stone disease within 2-4 years. Thus, the development of a novel ureteroscope/nephroscope capable of thoroughly removing all fragments from the kidney would be a welcome advance.

### SUMMARY OF THE INVENTION

Various embodiments include a device, comprising an optical instrument operably connected to a vacuum channel between 1.5 mm and 8.0 mm in width. In another embodiment, the vacuum channel is between 1.5 mm and 2.0 mm in width. In another embodiment, the optical instrument is an endoscope. In another embodiment, the optical instrument is a nephroscope. In another embodiment, the optical instrument is a ureteroscope. In another embodiment, the vacuum channel is the inner part of a vacuum scope. In another embodiment, the device further comprises a plurality of irrigation channels. In another embodiment, the plurality of irrigation channels are arrayed around the vacuum channel. In another embodiment, the plurality of irrigation channels include channels that are straight and angled. In another embodiment, the vacuum channel is adapted to capture a stone between 1 mm and 3 mm. In another embodiment, the vacuum channel is adapted for a suction form of biopsy of a polyp and/or tumor in a subject. In another embodiment, the vacuum channel is adapted for suction in a colon or a gastrointestinal of a subject. In another embodiment, the vacuum channel is a large bore central vacuum channel. In another embodiment, the vacuum channel is between 2.0 mm and 4.0 mm for use of the device as a ureteroscope. In another embodiment, the vacuum channel is between 6.0 mm to 8.0 mm for use of the device as a percutaneous nephroscope. In another embodiment, a visualization is achieved through an array of optical materials located around the vacuum channel. In another embodiment, a manipulation is achieved through a control mechanism located around the vacuum channel. In another embodiment, the manipulation further comprises a magnetic guide wire steering. In another embodiment, the device has a self-contained battery pack. In another embodiment, the device is Bluetooth and/or Wi-Fi enabled.

Other embodiments include a method of removing a biopsied tissue or stone particle or fragment thereof from an individual, comprising providing a device comprising an optical instrument operably connected to a vacuum channel between 1.5 mm and 8.0 mm in width, and removing the biopsied tissue or stone particle or fragment thereof from the individual. In another embodiment, the biopsied tissue or stone particle or fragment thereof is removed as part of a bronchoscopy, gastroscopy, duodenoscopy, small bowel endoscopy, colonoscopy, arthroscopy, and/or laryngoscopy procedure. In another embodiment, the biopsied tissue or stone particle or fragment thereof is removed as part of a ureteroscopic and/or percutaneous nephrolithotomy procedure. In another embodiment, the vacuum channel is between 1.5 mm and 2.0 mm in width. In another embodiment, a visualization is achieved through an array of optical materials located around the vacuum channel.

Other embodiments include a method of performing a biopsy in a subject, comprising providing a device comprising an optical instrument operably connected to a vacuum channel between 1.5 mm and 8.0 mm in width, and removing a biopsy sample from the subject. In another embodiment, the sample is a tumor and/or polyp. In another embodiment, the vacuum channel is between 2.0 mm and 8.0 mm.

Other features and advantages of the invention will become apparent from the following detailed description, taken in conjunction with the accompanying drawings, which illustrate, by way of example, various embodiments of the invention.

### DESCRIPTION OF THE DRAWINGS

FIG. 1 depicts, in accordance with embodiments herein, a modular wireless vacuum scope shown with a removal scope body and potentially wireless handpiece. In accordance with embodiments herein, the figure depicts a disposable (or reusable if desired) vacuum scope with connections for suction, fluid irrigation, laser fibers, and electrical contacts for CCD video as well as white light illumination, shown detached from the endoscope's handle.

FIG. 2 depicts, in accordance with embodiments herein, an example of a joystick magnetic guide wire steering mechanism that allows for 360 degree actuation of a ureterscope, depicting both an outside view as well as an inside view of the mechanism. The figure depicts a mechanism with an interfacing mechanism 101 between a wire and ball joint joystick, which is further connected to magnets in a cylindrical track 102, which in turn are connected to the tip of the scope 103. This mechanism involves the use of a joystick thumb control that is attached to several guy wires in the hand piece body. In accordance with various embodiments herein, this control mechanism couples to detachable and disposable endoscope tips through magnets. From this interface, the magnets embedded in the endoscope back end may then translate the pulling force to the distal tip of the scope deflecting the scope in the direction that tension is applied.

FIG. 3 depicts, in accordance with embodiments herein, a fluid optical wave guide.

FIG. 4 depicts, in accordance with embodiments herein, an example of implementation of a fluid optical wave guide.

FIG. 5 depicts, in accordance with embodiments herein, an example of a larger vacuum scope with an embedded 1.2 mm CMOS.

FIG. 6 depicts, in accordance with embodiments herein, an example of a 3D printed scope tip. FIG. 6(A) depicts a front perspective of the 3D printed scope tip. FIG. 6(B) depicts a diagonal perspective of the 3D printed scope tip.

FIG. 7 depicts, in accordance with embodiments herein, an example of a 3D printed scope tip. FIG. 7(A) depicts an isometric view of the 3D printed scope tip. FIG. 7(B) depicts a back view of the 3D printed scope tip.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As used herein, “treatment” or “treating” should be understood to include any indicia of success in the treatment, alleviation or amelioration of an injury, pathology or condition. This may include parameters such as abatement, remission, diminishing of symptoms, slowing in the rate of degeneration or decline, making the final point of degeneration less debilitating; improving a patient's physical or mental well-being; or, in some situations, preventing the onset of disease.

The terms “patient” and “subject” are used interchangeably and refer to patients and subjects of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

As used herein, the term “fluid optical wave guide,” also includes liquid based light waveguide.

As disclosed herein, current ureteroscopes have a working channel that is </=1.2 mm (<3.6 French) and are not equipped to provide suction evacuation of stone fragments. The small debris following laser fragmentation of a stone settles in the dependent calyces of the kidney and serves as a nidus for new stone formation, thus contributing to the recurrence of kidney stones after treatment and an ongoing cycle of treatment and recurrence. None of the currently available stone baskets can reliably remove fragments that are <1.5 mm. At this time, reliable complete stone clearance is only achieved with laparoscopic surgery, open surgery or with percutaneous stone removal; each of these is far more invasive than ureteroscopy. Thus, in accordance with an embodiment herein, the inventors have developed a universal endoscope that can serve as a ureteroscope or with a larger lumen as a nephroscope, with a specific vacuum channel that is, in some iterations, twice the size of current working channels. This endoscope should be able to evacuate all fragments that are 2 mm or smaller.

Furthermore, as known in the art, the infection rate after ureteroscopic lithotripsy is 8.4%, with common risk factors for infection including stone size, operative duration, and retained fragments that harbor bacteria. There are also challenges with regard to sterilization of the standard ureteroscope. Indeed, the sterilization process itself is injurious to the endoscope and limits its overall lifespan. There are no guidelines or quality benchmarks specific to ureteroscope reprocessing, and patient injuries and infections have been linked to the ureteroscope itself. Thus, in accordance with an embodiment herein, the invention provides a novel ureteroscope that is a disposable, single use endoscope, thus eliminating any problems and the costs due to reprocessing.

Further, in another embodiment, the invention comprises a true wireless or modular endoscope, wherein the handle, either disposable or sterilizable, of one endoscope (e.g. ureteroscope) could be used as the handle for another endoscope (e.g. gastroscope), thus decreasing costs. Further, the currently available endoscopes are tethered to large camera towers and to bulky display screens. Thus, in accordance with various embodiments herein, a truly wireless and modular endoscope would fill this gap of technology.

In one embodiment, the present invention provides a sterile, disposable or re-useable ureteroscope that integrates at minimum 6 French to 21 French vacuum/working (Vacuumoscopy) channel to ensure removal of virtually all stone particle debris at the end of a laser ablation of a renal stone with an ureteroscope or percutaneous nephroscopes, respectively. In another embodiment, the present invention expands indications for ureteroscopic laser stone fragmentation to stones larger than 1 cm. In another embodiment, the present invention provides a wireless and modular universal endoscope that could be used for bronchoscopy, gastroscopy, colonoscopy, and laryngoscopy to reduce costs of usability and simultaneously make endoscopy more portable.

In another embodiment, the invention will be used in all ureteroscopic or percutaneous nephrolithotomy laser lithotripsy procedures in which a kidney stone is broken into smaller fragments. Typical ureteroscopes have an approximate 1.2 mm channel that does not provide adequate suction to remove debris following stone laser fragmentation; indeed this same channel is used for irrigation for visualization within the kidney. These shortcomings will be overcome, in accordance with embodiments herein, by utilizing a larger bore central vacuum channel (2.5 mm or 7 French), with ongoing irrigation coming from smaller channels arrayed around the central vacuum channel. The endoscope will vary in length and diameter (French) depending upon the intended use (e.g. ureteroscopy vs. nephroscopy). During ureteroscopy procedures, visualization and manipulation of the endoscope can be achieved through an array of materials around the central vacuum channel (i.e. LED light, CCD imaging chip, guy wires for tip deflection, irrigation channels, and angled channels for laser and stone basket use). In another embodiment, a version of the novel disposable ureteroscope would also include a unique ergonomic handle allowing for one handed operation for advancing/retracting instruments, activating/deactivating irrigant flow, activating/deactivating suction, and a 360° thumb-driven joystick for universal tip deflection. Also, in another embodiment, the endoscope has a self-contained battery pack and is Bluetooth/Wi-Fi enabled thereby eliminating all electrical cords and cables. In another embodiment, the suction and irrigation tubing come off of the endoscope at 6 o'clock and directly posterior (i.e. 90 degrees to the handle), away from the surgeon's hands thereby adding to the ease of use and balance of the endoscope.

Other embodiments include a method of treating a condition or disease related to kidney stone removal in a subject, providing a device comprising a vacuumscope with a large bore central vacuum channel, and using the device as part of the removal and/or breakdown into smaller pieces of a kidney stone in a subject. In another embodiment, the device has a self-contained battery pack. In another embodiment, the device is Bluetooth and/or Wi-Fi enabled. In another embodiment, the device is a universal endoscope. In another embodiment, the device ensures removal of stone particle debris at the end of a laser ablation of a renal stone. In another embodiment, the device provides a wireless and modular universal endoscope. In another embodiment, the device may be used for nephroscopy, bronchoscopy, gastroscopy, colonoscopy, arthroscopy, and laryngoscopy. In another embodiment, the device is used as part of a ureteroscopic and/or percutaneous nephrolithotomy procedure in which a kidney stone is broken into smaller fragments.

In another embodiment, the present invention provides a device comprising a vacuum channel adapted for a suction form of biopsy of a polyp and/or tumor in a subject. In another embodiment, the vacuum channel is adapted for suction in a colon or the upper gastrointestinal tract (i.e. esophagus, stomach, duodenum, small bowel) of a subject.

In another embodiment, the present invention provides a device, comprising a vacuumscope with a large bore central vacuum channel. In another embodiment, the large bore central vacuum channel is between 2.5 mm to 7 mm for a ureteroscope or nephroscope respectively. In another embodiment, the large bore central vacuum channel is between 1.5 mm to 2.0 mm. In another embodiment, the large bore central vacuum channel is between 1.5 mm and 8.0 mm. In another embodiment, further comprising ongoing irrigation coming from smaller channels arrayed around the large bore central vacuum channel. In another embodiment, the vacuumscope provides a universal endoscope handle. In another embodiment, the device is used for ureteroscopy. In another embodiment, the device is used for nephroscopy. In another embodiment, visualization and manipulation is achieved through an array of materials around the large bore central vacuum channel such as LED light, CCD imaging chip, guy wires for tip deflection, irrigation channels, and straight or angled channels for laser and stone basket use. In another embodiment, further comprising a disposable unit. In another embodiment, the device provides for a reusable ureteroscope. In another embodiment, the device provides for a disposable ureteroscope. In another embodiment, the device comprises an ergonomic handle allowing for one handed operation for advancing and/or retracting instruments, activating and/or deactivating irrigant flow, activating and/or deactivating suction, and a 360 degree thumb-driven joystick for tip deflection. In another embodiment, further comprising an endoscope with a self-contained battery pack and/or Bluetooth and/or Wi-Fi enabled to eliminate electrical cords and cables.

In one embodiment, the present invention is a vacuumscope comprised of two single-time use disposable or potentially reusable units, such as the large vacuum endoscope removal tip and wireless and modular battery powered hand piece. The endoscope's shaft can vary (12 French to 26 French) with a varying central vacuum channel depending on the intended use (7 French to 21 French).

Other embodiments include a vacuumscope, comprising a large vacuum endoscope removal tip, and a wireless and modular battery powered hand piece. In another embodiment, the large vacuum endoscope removal body (i.e. handpiece) is disposable. In another embodiment, the large vacuum endoscope removal body (i.e. handpiece) is reusable. In another embodiment, the endoscope shaft is between 12 French and 26 French and may be either disposable or reusable. In another embodiment, the vacuumscope comprises a central vacuum channel between 7 French and 21 French.

Other embodiments include an overall treatment regimen in a subject, comprising providing a device comprising a vacuumscope with a large bore central vacuum channel, and using the device as part of a method for the removal and/or breakdown into smaller pieces of a kidney stone in a subject, wherein the method is part of a general surgical procedure on the subject.

In conjunction with various embodiments herein, devices relate to generally to the medical field, and more specifically, ureteroscopy and percutaneous nephrolithotomy. However, various embodiments herein are in no way limited to only ureteroscopy, and also may be used regarding, for example, laser ablation of ureteral and renal stone, capture and removal of stone fragments, intrarenal and intraureteral surgical procedures (e.g. endoureterotomy, endopyelotomy, tumor resection), and biopsy of urothelial based renal and ureteral lesions.

### EXAMPLES

### Example 1

### Generally

In accordance with various embodiments herein, the inventors developed a sterile, disposable or re-useable ureteroscope that integrates at minimum 6 French to 26 French vacuum/working (Vacuumoscopy) channel to ensure removal of all stone particle debris at the end of a laser ablation of a renal stone after ureteroscope (i.e. 6F channel) or percutaneous nephrolithotomy (up to 21F). Further, the device may expand indications for ureteroscopic laser stone fragmentation to stones larger than 1 cm. In another embodiment, the device is a wireless and modular universal endoscope that could be used for bronchoscopy, gastroscopy, colonoscopy, arthroscopy, and laryngoscopy to cut down on costs of usability and make endoscopy more portable.

### Example 2

### Overview

In one embodiment, the invention will be used in all ureteroscopic or percutaneous nephrolithotomy laser lithotripsy procedures in which a kidney stone is broken into smaller fragments. Typical ureteroscopes have an approximate 1.2 mm channel that does not provide for an adequate channel to suction and remove debris following stone laser fragmentation; indeed this same channel is used for irrigation for visualization within the kidney. The invention will overcome such shortcoming by utilizing a larger bore central vacuum channel (2.5 mm to 7 mm) with a drainage connection that does not compromise the endoscope's central vaccum channel; with ongoing irrigation coming from smaller channels arrayed around the central vacuum channel. The vacuumscope will vary in length and diameter (French) dependent upon the intended use. During ureteroscopy procedures, visualization and manipulation of the endoscope will be achieved through an array of materials around the central vacuum channel (i.e. LED light, CCD imaging chip, guy wires for tip deflection, irrigation channels, and straight/angled channels for laser and stone basket use). A version of the novel disposable ureteroscope would also include a unique detachable ergonomic handle allowing for one handed operation for advancing/retracting instruments, activating/deactivating irrigant flow, activating/deactivating suction, and a 360° thumb-driven joystick for tip deflection. Also, the endoscope has a self-contained battery pack and is Bluetooth/Wi-Fi enabled thereby eliminating all electrical cords and cables. The suction and irrigation tubing comes off of the endoscope at 6 o'clock and directly posterior (i.e. 90 degrees to the handle), away from the surgeon's hands thereby adding to the ease of use and balance of the endoscope.

In one embodiment, the Vacuumscope invention is comprised of two single-time use disposable or potentially reusable units namely the large vacuum endoscope removal shaft and the wireless and modular battery powered hand piece. The endoscope's shaft will vary (12 French to 26 French) with a varying central vacuum channel depending on the intended use (7 French for ureteroscopy to 21 French for percutaneous nephroscopes).

In accordance with various embodiments herein, one may increase the range of deflective for an endoscope tip incorporating fluid wave guide, as the fluid wave guide is more malleable than a fiberoptic light guide thereby making the tip of the endoscope easier to deflect. The liquid waveguide would with the appropriate difference in refractive index between core and cladding could withstand a tighter bend radius than would a rigid solid such as borosilicate glass used in fiber optics. Furthermore, depending upon the variation of the two refractive indices, one could manipulate the angle at which the light energy would undergo total internal reflection that would further provide a means increasing the range of deflective for the scope tip.

### Example 3

### Some Advantages

At minimum one large bore (i.e. 7 French to 21 French) vacuum port to evacuate all stone fragments <1.5 mm after ureteroscopy or larger remnants from laser lithotripsy via a percutaneous nephroscope.
Disposable thereby eliminating risks of infection or endoscope malfunction due to processing.
Wireless video capabilities via Bluetooth and/or Wi-Fi thereby eliminating entangling cords and expensive high power light sources and fixed camera equipment.
Superior universal 360 degree deflection based on joystick steering—eliminating the need for the surgeon to employ excessive wrist movement to guide the tip of the endoscope.
Modular design capable of adapting various endoscope shafts to attach to the same universal handle.
Unique ergonomic handle design allowing for single-handed control of irrigation, suction, tip deflection, and instrument proj ection/retraction.

### Example 4

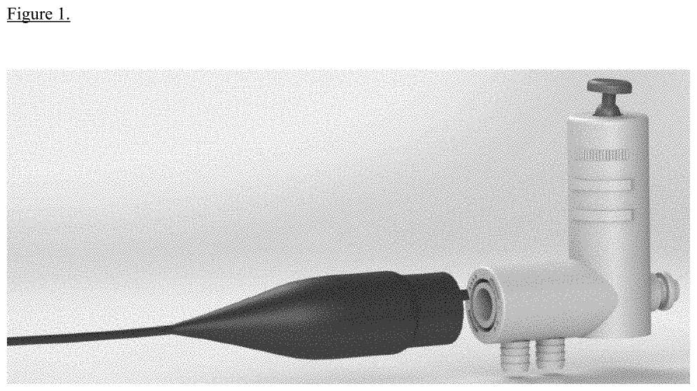

### FIGS. 1-7 Herein in Greater Detail

Turning to FIGS. 1-7 herein in greater detail, in accordance with various embodiments herein, the inventors have developed several novel and effective versions of endoscopes with vacuum channels.

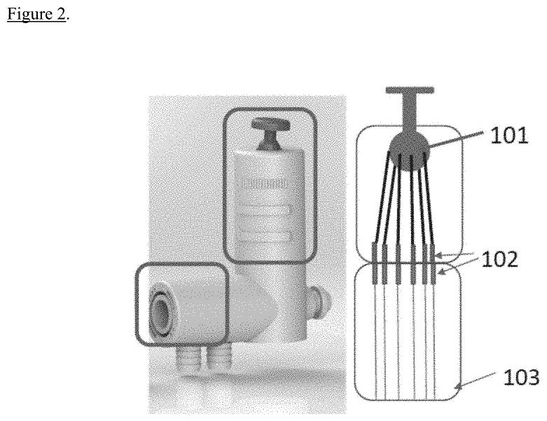

For example, as referenced in FIG. 2, the inventors have developed a mechanism for 360 degree actuation of a ureterscope that allows for more intuitive navigation through the urinary tract anatomy. This mechanism involves the use of a joystick thumb control that is attached to several guy wires in the hand piece body as seen in the schematic in FIG. 2 herein. This said control mechanism couples to detachable and disposable endoscope tips through magnets that can be seen in the schematic at the end of the hand piece. From this interface, the magnets embedded in the endoscope back end will then translate the pulling force to the distal tip of the endoscope deflecting the endoscope in the direction that tension is applied. Alternatively, in a commercially available ureteroscope, there is only a two way actuation that provides 180 degrees of actuation. In this alternative commercially available ureteorscope, the user is forced to rotate the endoscope or supinate his/her wrist in order to steer the endoscope into the various infundibula and calyces of the renal collecting system. As referenced in FIG. 2 herein, and in accordance with various embodiments herein, the inventors have developed a design that provides a straightforward and intuitive approach to solving the problem of having to rotate the endoscope that is otherwise presented by commercially available alternatives.

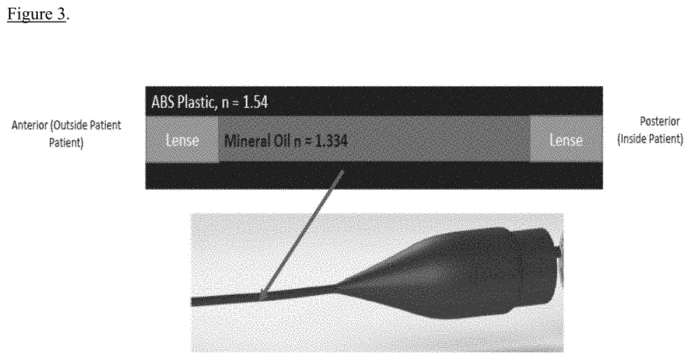

As referenced in FIG. 3 herein, and in accordance with various embodiments herein, the inventors have developed a vacuum ureteroscope with a plurality of mechanisms to transmit light energy, for example, either for illumination or for relaying visible light to render an image. FIG. 3 details the use of a liquid based light waveguide, or fluid optical wave guide. Such a waveguide would transmit light energy through total internal reflection a process governed by the change in refractive index between the waveguide and the surrounding material. At both ends of said liquid wave guide there will be a lens focusing optics to transmit the relayed light. In this proposed example mineral oil is shown as the inner waveguide liquid surround by ABS plastic. Fiber optic glass or silicon dioxide waveguides are more typical for such applications. This waveguide will permit the design of endoscope to embody a larger suction port than typically permitted in endoscopes today. Video endoscopes port configuration is limited by the size of the CMOS chip that is present at the distal end of the scope.

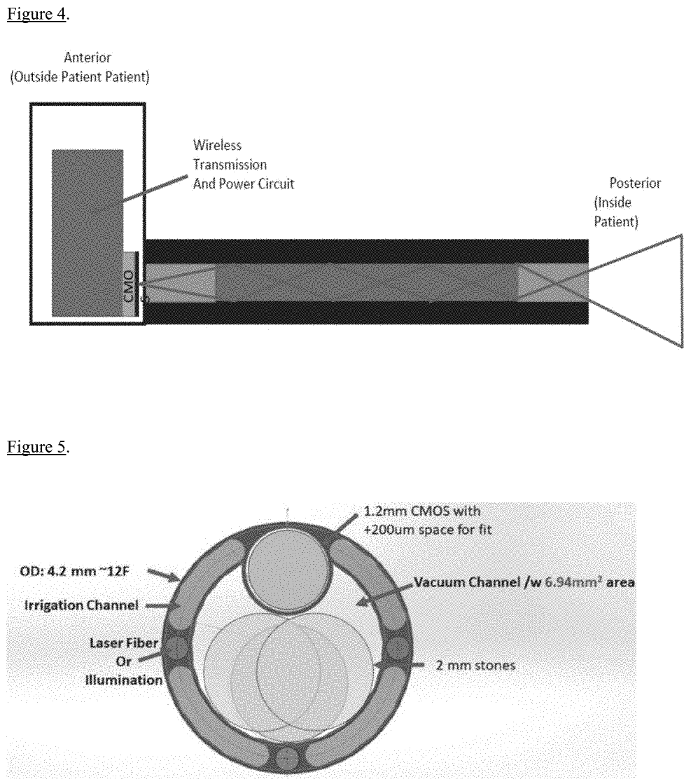

As referenced in FIG. 4 herein, and in accordance with various embodiments herein, an example of an imaging system can be seen in the figure. FIG. 4 describes the implementation of said liquid light wave guide. On the anterior portion of the device assembly, there is a CMOS or CCD imaging chip. On the distal or posterior portion of the device, there is the liquid light wave guide. The two could be coupled with a lens to focus the image relayed from the liquid waveguide to the CCD or CMOS chip in the handle. This aspect of the design provides more area on the endoscope tip for a vacuum port since the imaging aspect of the device is being moved more towards the hand piece, where there is greater area. With a larger suction port, the device will be able to suction larger stones.

FIG. 5 herein, and in accordance with various embodiments herein, illustrates an example of a configuration of an endoscope to be used with a 1.2 mm CMOS camera on the distal end, rather than in the handle. In this example, the outer diameter of the scope is restrained to a 12-14 Fr range. This limitation is posed through the size of the ureter with an access sheath placed into the ureter; while the largest access commercially available is 16 Fr (with a 14 Fr lumen), there is the possibility that larger access sheaths may come into existence such that a 16 Fr or 18 Fr or 20 Fr ureteroscope will be used with a corresponding 18 Fr, 20 Fr, or 22 Fr ureteral access sheath. With such a geometry presented in the figure, one potential targeted stone fragment diameter that may be picked up with a ureteroscope is 2 mm, far greater than what is typical possible with conventional endoscopes given the limited size of the 1.2 mm working port. In addition, alternative commercially available endoscopes do not often provide irrigation and suction independently, which hinders the ability to catch all stone fragments efficiently as the single channel can only be used for irrigation or suction at a given time. In the proposed endoscope, active irrigation via the outer channels can continue while suction is applied via the larger central vacuum port.

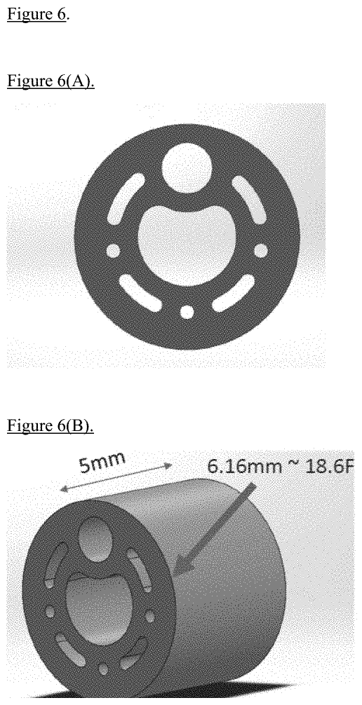

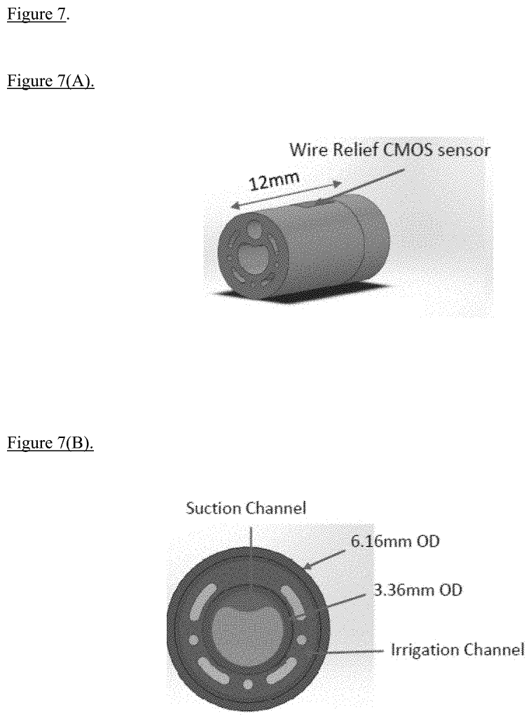

FIGS. 6 and 7 herein describe an example of a 3-D printed scope tip developed by the inventors. FIG. 7 depicts the labeled back end of the scope tip where relevant suction and irrigation can be applied. In one embodiment, the probe tip is the most distal portion of the device, due to impracticality of extruding tubing.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A device, comprising:
an optical instrument operably connected to a vacuum channel between 1.5 mm and 8.0 mm in width.

2. The device of claim 1, wherein the vacuum channel is between 1.5 mm and 2.0 mm in width.

3. The device of claim 1, wherein the optical instrument is an endoscope.

4. The device of claim 1, wherein the optical instrument is a nephroscope.

5. The device of claim 1, wherein the optical instrument is a ureteroscope.

6. The device of claim 1, wherein the vacuum channel is the inner part of a vacuum scope.

7. The device of claim 1, further comprising a plurality of irrigation channels.

8. The device of claim 7, wherein the plurality of irrigation channels are arrayed around the vacuum channel.

9. The device of claim 7, wherein the plurality of irrigation channels includes channels that are straight and angled.

10. The device of claim 1, wherein the vacuum channel is adapted to capture a stone between 1 mm and 3 mm.

11. The device of claim 1, wherein the vacuum channel is adapted for a suction form of biopsy of a polyp and/or tumor in a subject.

12. The device of claim 1, wherein the vacuum channel is adapted for suction in a bronchus, colon or any area of the gastrointestinal tract of a subject.

13. The device of claim 1, wherein the vacuum channel is a large bore central vacuum channel.

14. The device of claim 1, wherein the vacuum channel is between 2.0 mm and 4.0 mm for use of the device as a ureteroscope.

15. The device of claim 1, wherein the vacuum channel is between 6.0 mm to 8.0 mm for use of the device as a percutaneous nephroscope.

16. The device of claim 1, wherein a visualization is achieved through an array of optical materials located around the vacuum channel.

17. The device of claim 1, wherein a manipulation is achieved through a control mechanism located around the vacuum channel.

18. The device of claim 17, wherein the manipulation further comprises a magnetic guide wire steering.

19. The device of claim 1, wherein the device has a self-contained battery pack.

20. The device of claim 1, wherein the device is Bluetooth and/or Wi-Fi enabled.

21. A method of removing a biopsied tissue or stone particle or fragment thereof from an individual, comprising:
providing a device comprising an optical instrument operably connected to a vacuum channel between 1.5 mm and 8.0 mm in width; and
removing the biopsied tissue or stone particle or fragment thereof from the individual.

22. The method of claim 21, wherein the biopsied tissue or stone particle or fragment thereof is removed as part of a bronchoscopy, gastroscopy, duodenoscopy, small bowel endoscopy, colonoscopy, arthroscopy, and/or laryngoscopy procedure.

23. The method of claim 21, wherein the stone particle or fragment thereof is removed as part of a ureteroscopic and/or percutaneous nephrolithotomy procedure.

24. The method of claim 21, wherein the vacuum channel is between 1.5 mm and 2.0 mm in width.

25. The method of claim 21, wherein a visualization is achieved through an array of optical materials located around the vacuum channel.

26. A method of performing a biopsy in a subject, comprising:
providing a device comprising an optical instrument operably connected to a vacuum channel between 1.5 mm and 8.0 mm in width; and
removing a biopsy sample from the subject.

27. The method of claim 26, wherein the sample is a tumor and/or polyp.

28. The method of claim 26, wherein the vacuum channel is between 2.0 mm and 8.0 mm.
